(19) United States
(12) Patent Application Publication
(10) Pub. No.: US 2003/0181982 A1
(43) Pub. Date: Sep. 25, 2003

(54) No-profile, lumbo-sacral fixation device and method

(75) Inventor: Stephen D. Kuslich, Stillwater, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Spineology, Inc.

(21) Appl. No.: 10/379,426

(22) Filed: Mar. 4, 2003

Related U.S. Application Data
(60) Provisional application No. 60/361,616, filed on Mar. 4, 2002.

(51) Int. Cl. A61F 2/44

(52) U.S. Cl. 623/17.16; 623/17.11

(57) ABSTRACT

A method and apparatus for stabilizing the lumbo-sacral junction using an upper bone anchor interfaced with the L5 vertebra and a lower bone anchor interfaced with the S1 sacral bone. An intermedullary rod connects the upper and lower bone anchors. Preferably, the intermedullary rod is angled at an angle relative to a longitudinal axis of the spinal column that replicates a desired angle between the L5 vertebra and the S1 sacral bone. In one embodiment, a distal end of the lower bone anchor is secured into the S1 sacral bone such that a proximal end of the lower bone anchor does not protrude above an anterior surface of the S1 sacral bone. In another embodiment, the lower bone anchor has at least one variable angle socket adapted to receive a fastener to secure into the S1 sacral bone.

### RELATED APPLICATION

The present application claims the benefit of U.S. Provisional Application No. 60/361,616 filed Mar. 4, 2002, which is incorporated herein in its entirety by reference.

### FIELD OF THE INVENTION

The present invention relates generally to implantable prosthesis for spine bone. More specifically, the present invention relates to a fixation device and method for the lumbo-sacral junction along the vertebral column.

### BACKGROUND OF THE INVENTION

A wide variety of pathological conditions may cause the human spine to become unstable, i.e. unable to sustain physiologic loads without structural failure. Re-stabilization, by means of fixation and fusion of the vertebral column, is a long practiced surgical means for treating these conditions. The lumbo-sacral junction, also known as “L5-S1”, is that area of the skeletal anatomy where the spine is joined to the pelvis. Because of several anatomic peculiarities at L5-S1, devices and techniques that are useful in other areas of the spine may not be safe, or effective, or feasible at the lumbo-sacral junction.

Surgical approaches to the spine may be anterior (front), posterior (back) or lateral (side), or a combination of approaches. The L5-S1 junction is particularly difficult to stabilize, regardless of approach. The most commonly used approach is the posterior (back) approach. However, if the target pathology—e.g. tumor, fracture, or degenerative disc disease—is located in the anterior portion of the spine, an anterior (front) approach may be preferred or even essential for proper treatment. To gain access to the anterior portion of the L5-S1 junction from the posterior direction, the surgeon must pass through or around the nerves of the cauda equina. Damaging any of these nerves will result in serious permanent injury.

Approaching the L5-S1 junction from the direct lateral direction is practically impossible, because the ileum, the alar of the sacrum, and the L5 nerves block this approach. One can access the L5-S1 junction from an inferior direction (the pelvic approach, along the anterior border of the lower sacrum), but this approach is even more hazardous. A pelvic approach requires the surgeon to deal with all of the anatomic structures of the lower pelvis: the genitals, bladder, uterus, colon and a host of delicate nerves and blood vessels that affect functions of the urinary and reproductive systems. For these and other reasons, direct anterior approaches to L5-S1 are often preferred.

Certain anatomical features of the L5-S1 must be taken into account when designing fixation devices for that portion of the spine. The spinal canal (the space containing the lower spinal cord—the cauda equina) is narrow at the L5-S1 level, thus, bulky hardware fixation systems, requiring long bone screws for attachment, and installed from the anterior to posterior direction, might impinge on delicate nerves. The anterior-posterior length of S1 is quite short. Measured along its central axis, beginning at its most superior end, the anterior to posterior dimension of the sacrum, already smaller than other areas of the spine, decreases rapidly as one passes from the cephalic to the caudal position. This feature severely limits the amount of bone stock that is available for internal fixation devices. In other words, there is only a small amount of bone stock into which fixation devices can be embedded. The cortical bone of the anterior S1 wall is very thin. This bone will not support traditional screws and bolts, when physiological loads are repeatedly applied to the construct. Further, the sacrum is inclined backward from a line drawn through the long axis of the spine. This lumbo-sacral inclination (in the front-back plane) varies among people from about 10 to 35 degrees or more. This inclination requires that direct anterior fixation devices be precisely bent or angled. Bending a fixation device can produce cracks and crevices that reduce strength by promoting fatigue failure. Finally, body weight above L5, pressing downward against S1, causes shear across the L5-S1 junction. This shear tends to push anterior fixation devices off the top of the sacrum, or through the bone material of the anterior portion of the sacral vertebral body.

As a consequence of the above considerations, spinal fixation appliances that are appropriate and useful in other regions of the spine are usually not appropriate or useful at the L5-S1 level. Clearly, different forms of fixation device are needed at this level of the spine.

### DESCRIPTION OF RELATED ART

A variety of fixation devices are commonly used for spinal fixation. A representative list of the most popular anterior systems includes: Synthes anterior spinal plate, the University Plate, The Z-Plate and the Kaneda device. These systems are restricted in use to the lateral aspect of the spine, but because of the anatomical considerations listed in above paragraphs, they cannot be applied laterally at L5-S1. If one of these systems was to be inappropriately applied anteriorly at L5-S1, its bulkiness could cause great vessel damage and/or rupture, as occurred when a similar device, the Dunn Device, was used on the anterior surface of the spine.

A large variety of interbody cages and interbody spacers, e.g. BAK, Ray, Brantigan are available to surgeons. However, if the L5-S1 junction is highly unstable, as it often is, cages as stand-alone devices are inadequate. Therefore, surgeons must add another fixation device, such as a posterior pedicle fixation system, in order to regain stability. Implanting both the anterior cage or spacer, and posterior pedicle fixation system, adds a great deal of time, risk, morbidity and cost to the procedure. Some examples of cages and spacers include, but are not limited to J. Harms' “Harms Cage”, pedicle systems, femoral ring spacers such as have been described in papers by J. O'Brien, and posterior facet screws.

Other inventive techniques promote fully enclosed fixation devices (devices in which no part protrudes beyond the outer surface of the spine) as a solution. These inventive techniques include: the Bohlman method using a fibular graft and the Kuslich device and method for fixing spondylolisthesis from the posterior direction. These techniques, while useful in some cases, may not allow for direct anterior excision of the target pathology. Furthermore, they do not allow for correction of deformity (such as spondylolisthesis) prior to fixation.

The anterior spondylolisthesis system of Kuslich such as is described in U.S. Pat. No. 6,086,589, while useful in many circumstances, requires somewhat enlarged incision, due to the extended trajectory of the transferring component, and significant mobilization of the great vessels during implantation. Another Kuslich invention known as the K-Centrum® device(s) are described in U.S. Pat. No. 5,591,235. The K-Centrum® solves some of the problems listed above, however due to the tin cortex of S1 anteriorly, the K-Centrum® bone anchors may have less than the desired holding power. Unless modified, incorporating features of the current invention, fixation onto the S1 vertebral body may be inadequate.

In summary, all currently available fixation systems suffer from one or more of the following inadequacies when applied to the L5-S1 region. They are high profile systems, i.e., they are thick and bulky. If placed on the anterior surface of the spine, they would irritate or damage the great vessels, the aorta and vena cava. They are not stable or “standalone systems”. They require precise bending to fit the sacral inclination. They cannot be installed from the preferred direct anterior approach. They are cantilevered systems, i.e., they hold onto the spine from a position that is distant from the axis of motion. They do not provide large ingrowth vertebral anchors, and therefore fixation can loosen when repeatedly stressed by physiologic loads during the post-operative period. Finally, they require significant manipulation of the great vessels during implantation. Clearly, therefore, there is a need for an improved fixation system for the lumbo-sacral junction.

### SUMMARY OF THE INVENTION

The present invention is method and apparatus for stabilizing the lumbo-sacral junction using an upper bone anchor interfaced with the L5 vertebra and a lower bone anchor interfaced with the S1 sacral bone. An intermedullary rod connects the upper and lower bone anchors. Preferably, the intermedullary rod is angled at an angle relative to a longitudinal axis of the spinal column that replicates a desired angle between the L5 vertebra and the S1 sacral bone. In one embodiment, a distal end of the lower bone anchor is secured into the S1 sacral bone such that a proximal end of the lower bone anchor does not protrude above an anterior surface of the S1 sacral bone. In another embodiment, the lower bone anchor has at least one variable angle socket adapted to receive a fastener to secure into the S1 sacral bone

### BRIEF DESCRIPTION OF THE DRAWINGS

FIG. 1 is a perspective view of the invention having a lower anchor embodied as a walled enclosure.

FIG. 2 is a perspective view of the invention having a lower anchor embodied as a plate.

FIGS. 3-5 depict the sacral attachment means placed in the alar of the sacrum.

FIGS. 6-9 depict the lower anchor embodied as a walled enclosure.

FIGS. 10-21 depict the lower anchor embodied as a plate.

FIGS. 22-25 depict the lower anchor with a pivotal head for receiving the sacral attachment means.

FIGS. 26-29 are perspective views of the sacral attachment means placed in the lower anchor with a pivotal head.

### DETAILED DESCRIPTION OF THE INVENTION

The current invention provides solutions to the problems, concerns and difficulties described above by means of the following strategies and features. The inventive device is a “no-profile” device, i.e., once installed, no portion of the device lies outside the outer shell of the spinal bones. Because it is not placed on the surface of the spine, the device cannot irritate or damage local anatomic structures, especially the great vessels.

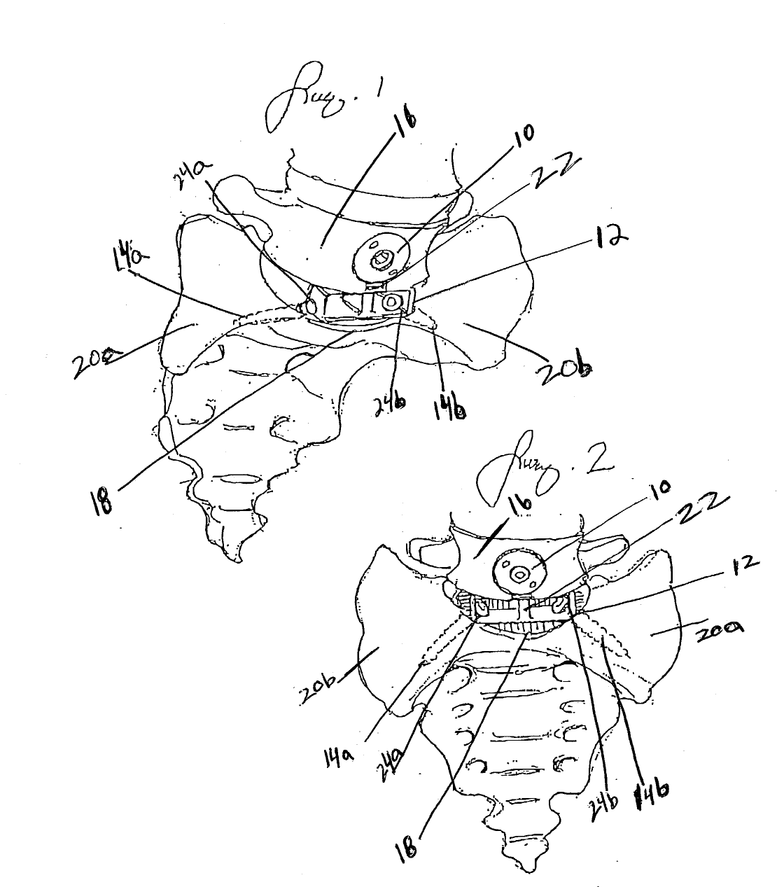

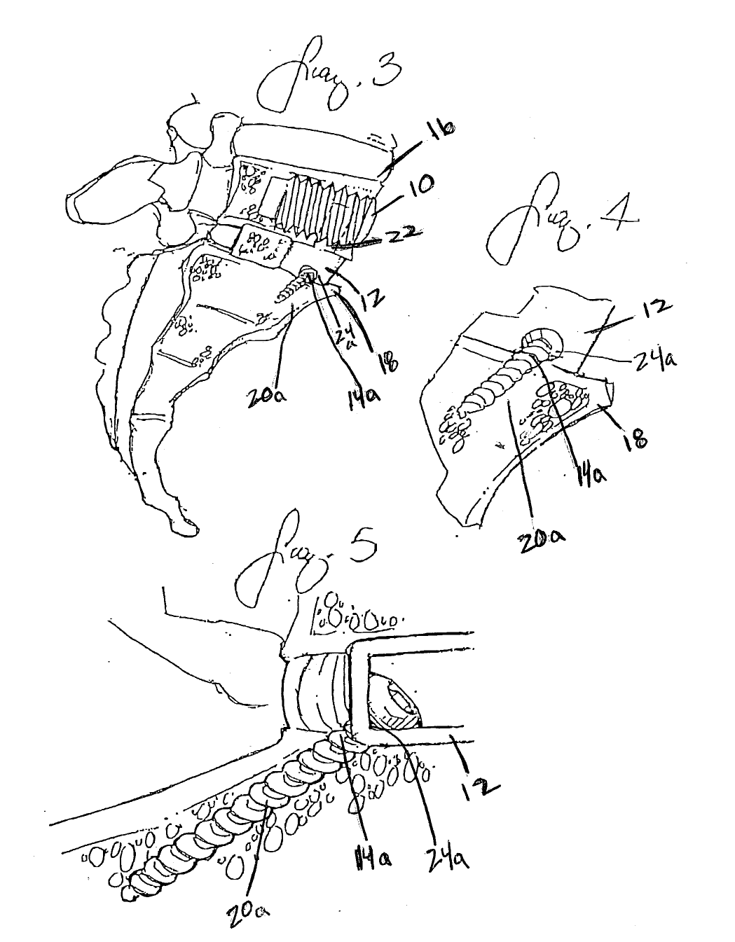

In preferred embodiments of the invention, as shown in FIGS. 1 and 2, an upper bone anchor 10 is placed in the last lumbar vertebra (L5) 16. A lower body anchor 12 includes sockets 24a and 24b that receive fasteners 14a and 14b interfacing the lower bone anchor 12 with the sacrum (S1) 18. An intermedullary rod 22 connects the upper bone anchor 10 to the lower bone anchor 12. The inventive device does not require precise bending to fit the sacral inclination. In one embodiment, it is manufactured and supplied in a variety of angle configurations. In another embodiment, variable angle screw placement sockets 24a and 24b allow screws to be placed at the appropriate angle.

The inventive device can be installed from the preferred direct anterior approach. Once installed, the construct, i.e., the combination of the device and the portion of the spine affixed to the device, is stable. It is therefore, a “standalone” device. No additional fixation devices are necessary.

The inventive device is not a cantilevered system. It is designed to be placed near the center of the axis of spinal motion. The inventive device does provide a large ingrowth vertebral anchor 10 at the last lumbar level. That anchor becomes more secure as it is incorporated into the structure of the spinal vertebral body bone. Under normal anatomic conditions, the inventive device can be installed with minimal manipulation of the great vessels.

The present invention provides a device that can be safely installed onto the L5-S1 junction using the anterior abdominal, trans-peritoneal or retro-peritoneal approaches. The anterior method of inserting the device provides a potentially safer, less obstructed access path to the L5-S1 junction than the posterior, lateral or pelvic approaches allow.

In summary, some of the unique features of the present design include: a large ingrowth anchor 10 for L5, a lower anchor 12, and an intermedullary rod 22 joining the upper anchor 10 to the lower anchor 22. The lower anchor-rod portion is supplied in a variety of angles to match the lumbo-sacral inclination. There may be variable angle sockets 24a and 24b in the lower-lateral aspect of the lower bone anchor 12. Cancellous alar bone screws 14a and 14b are directed through sockets 24a and 24b and into the ala 20a and 20b of the sacrum (the strongest portion of the sacral vertebral body). Once installed, the entire device is contained within the borders of the spine, i.e. it is “no profile”. The lower bone anchor 12 allows for the addition of interbody bone to aid in arthrodesis (fusion) of the L5-S1 junction.

The device applies its sacral attachment screws 14a and 14b adjacent to the anterior surface 18 of the sacrum. This region of the sacrum contains the strongest bone in the region. This portion of the sacral bone remains relatively strong, even in osteoporotic patients.

The device of the present invention includes a lower anchor 12 to carry bone loads. A rod 22 attached to the lower anchor 12 secures the lower anchor 12 to the upper bone anchor 10, such as a K-Centrum® bone anchor available from Spineology, Inc. At least one hole 24a in the lower bone anchor 12 allow screws 14a and 14b to be placed therethrough, interfacing the lower bone anchor 12 to the sacral bone 18.

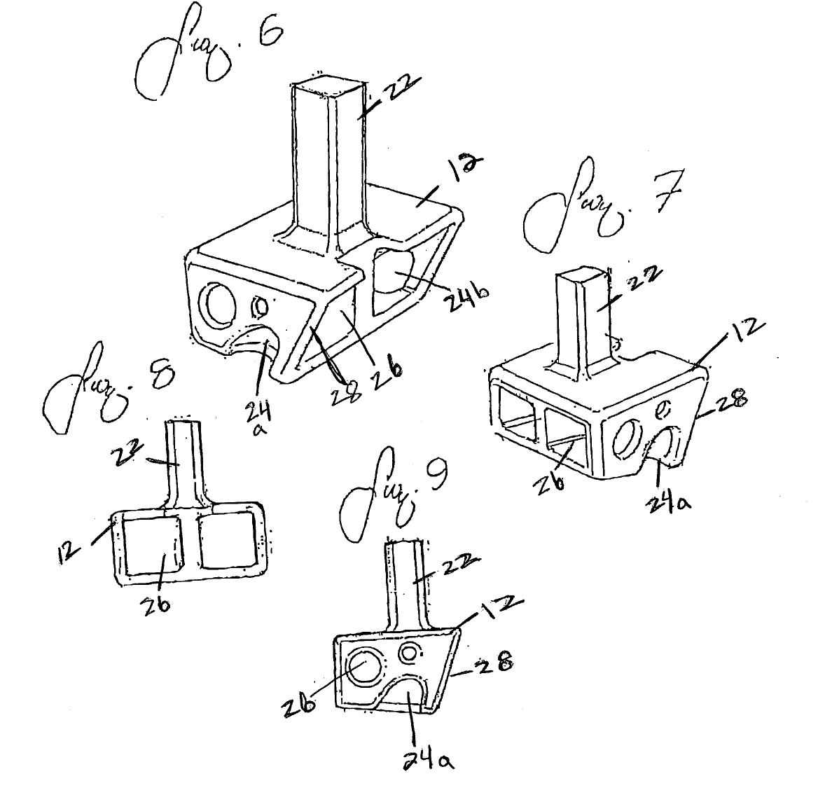

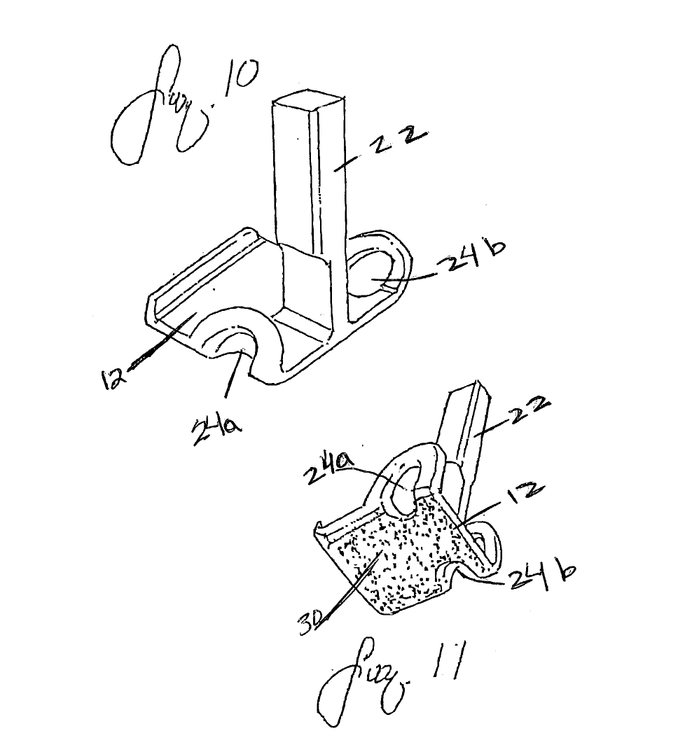

The lower bone anchor 12 may be configured in many ways. In one embodiment, as shown in FIGS. 6-9, the lower bone anchor 12 may be a six-walled enclosure, similar to cage. The rod 22 is attached to the first wall (the superior wall). The second wall (the inferior wall) is placed against the proximal (superior) end of S1. Screw holes 24a and 24b at the junction of the third and fourth walls (the lateral walls) and the inferior wall, allow for placement of the screws 14a and 14b into the alar portions of the sacral bone. The fifth and six walls (anterior and posterior walls) have large openings 26 that allow for the placement of fill material. Fill material may consist of bone graft, a bone graft substitute, polymethylmethacrylate or other bone cement, bone morphogenic protein, or any other bone growth stimulating substance. Any or all of the walls of lower bone anchor 12 may be angled 28 to mimic the degrees of sacral inclination and the trapezoidal shape of the longitudinal section outline of the L5-S1 joint. The lower bone anchor 12 transfers weight to the proximal end of S1 18, then to the screws 14a and 14b that are positioned against the very dense cortex of the anterior surface of the alar of the sacrum.

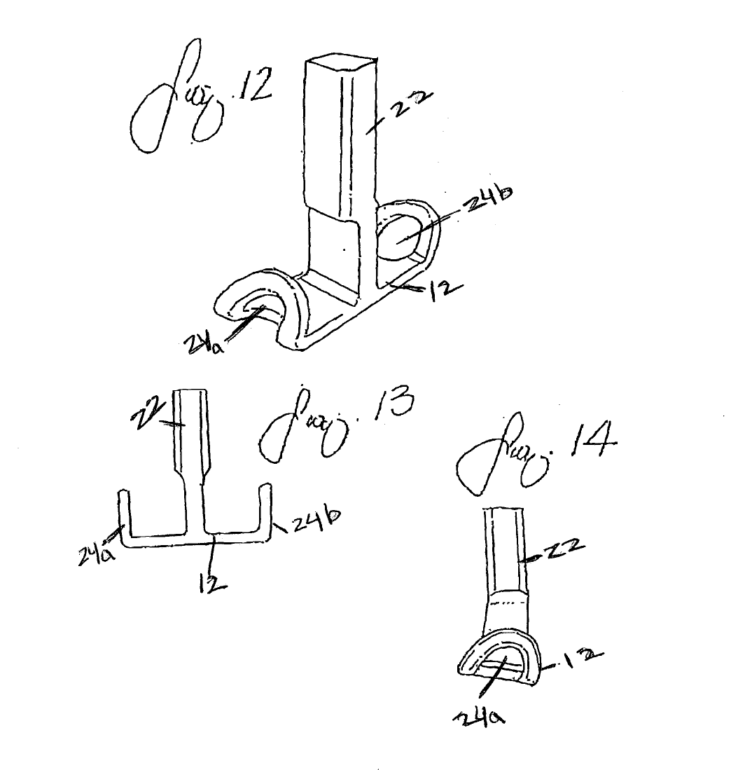

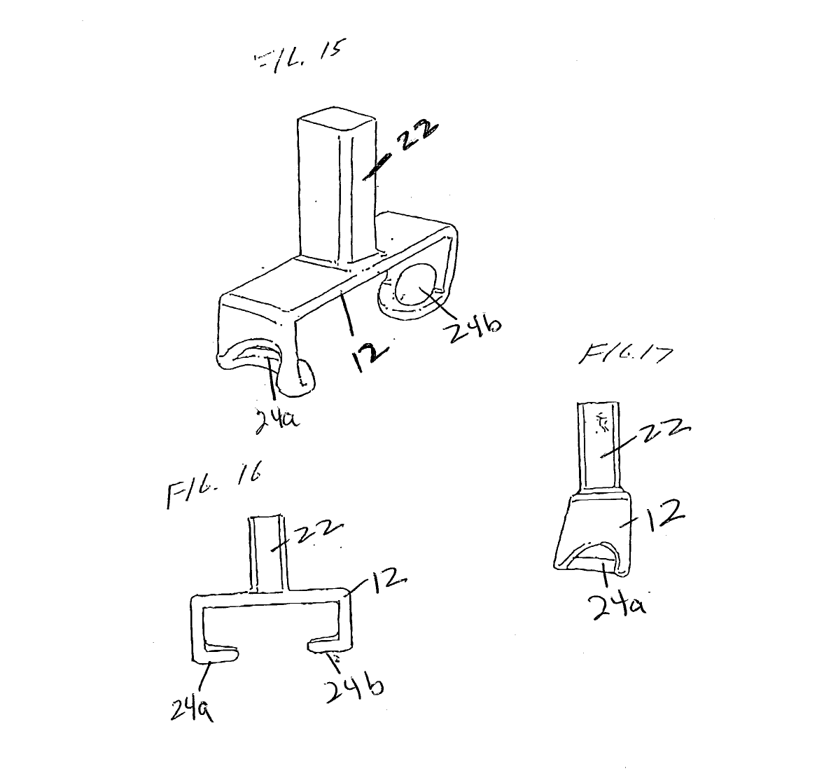

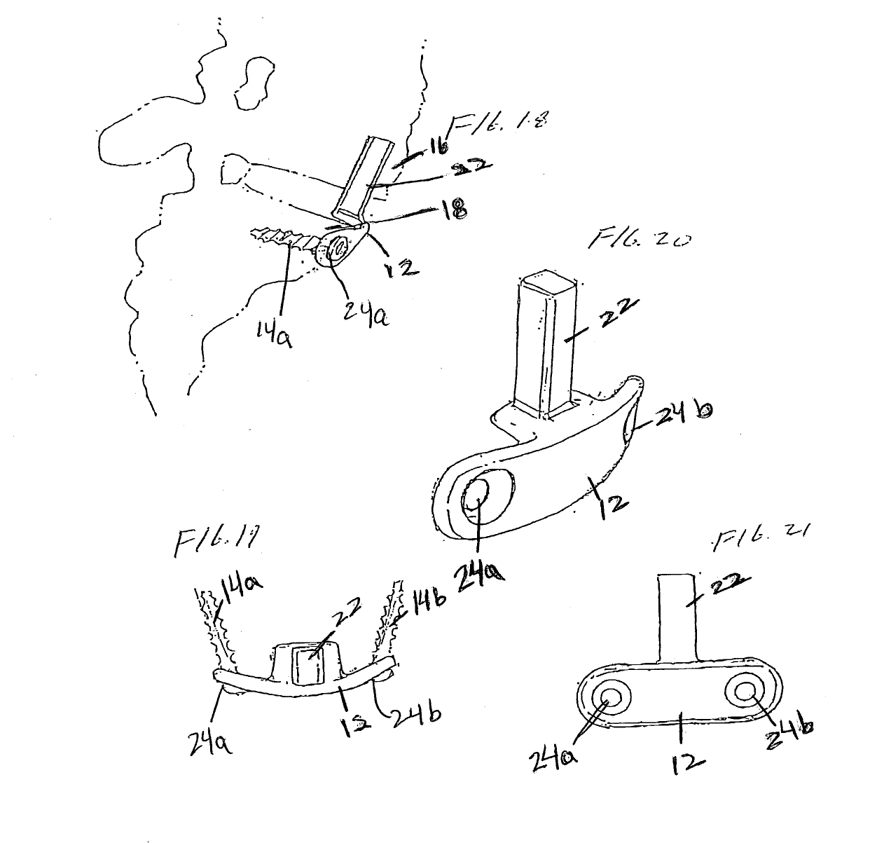

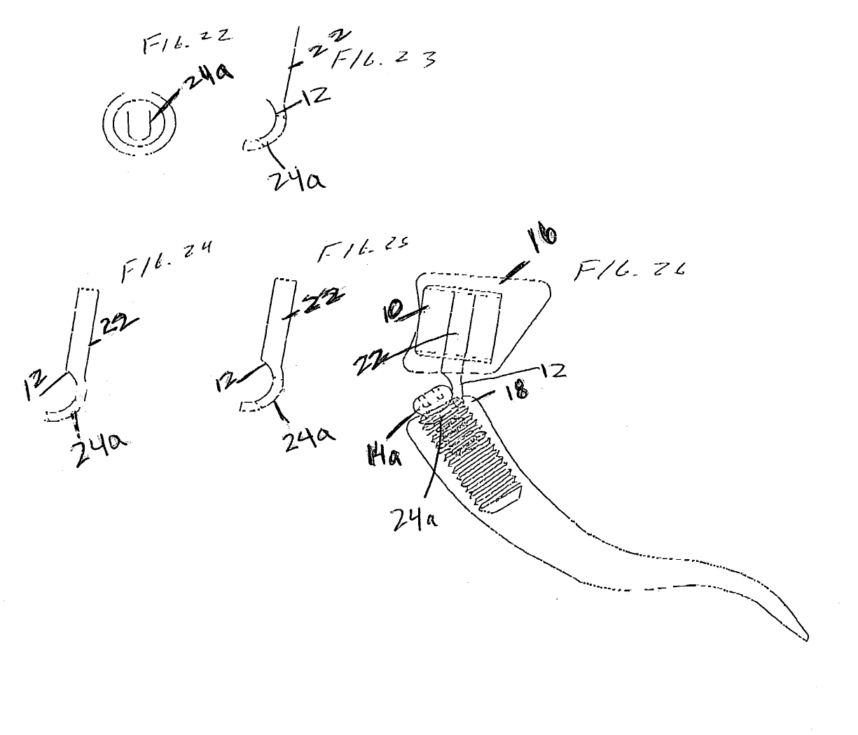

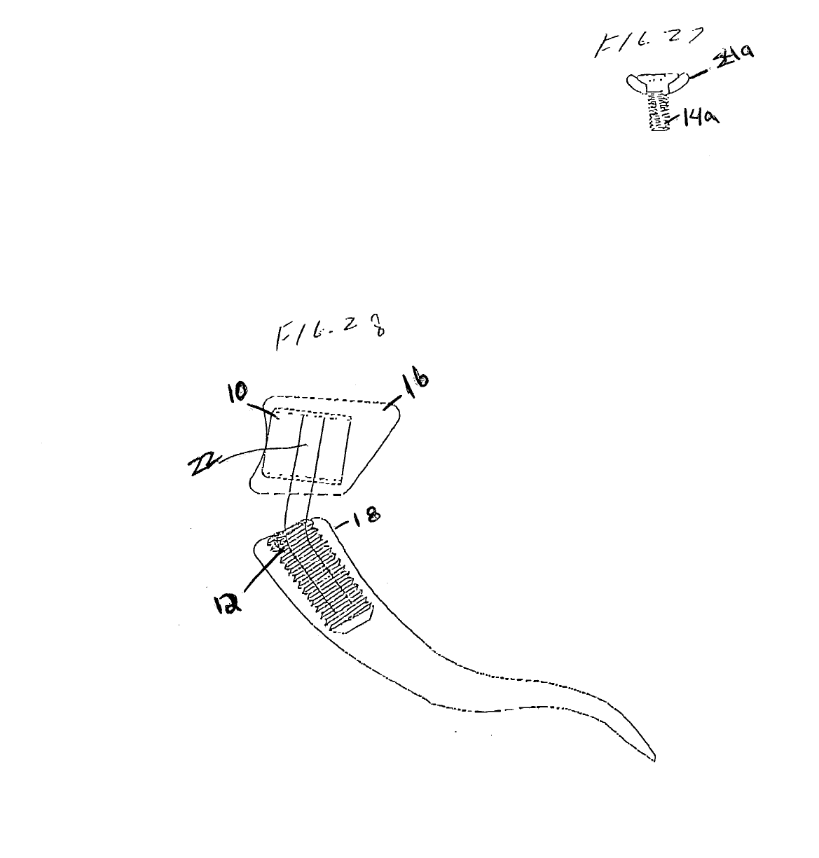

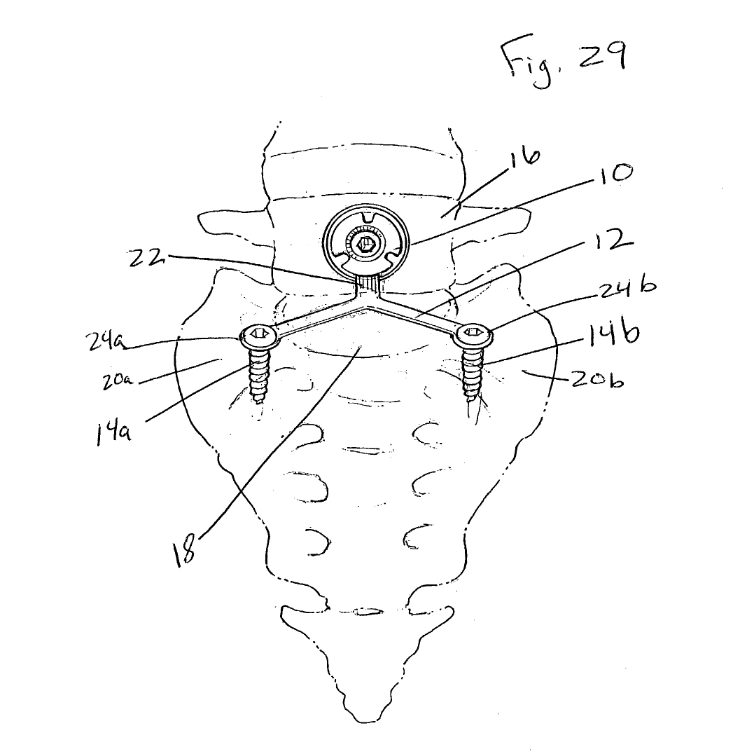

In another embodiment, as shown in FIGS. 10-21, the lower bone anchor 12 is simply a footplate, interfacing with the superior surface of the S1 18 vertebral body. In this case, the linkage rod 22 is affixed to the central region of the lower bone anchor 12, and the lower bone anchor 12 contains holes 24a and 24b for screw 14a and 14b attachment near the lateral regions of the lower bone anchor 12.

In yet another embodiment, the lower bone anchor 12 is a half cage, lacking anterior and posterior walls. This embodiment would provide less interference when x-rays are taken in the anterior-posterior plane.

As shown in FIG. 11, the surface of the lower bone anchor 12 may be roughened or otherwise treated to improve the interface with S1 18 or bone ingrowth potential. The surface of the lower bone anchor 12 may be porous or non-porous. The walls of the lower bone anchor 12 may be angled to correspond to the sacral inclinations of the patient's anatomy. The lower bone anchor 12 may have varying heights to correspond to the disc space height.

The method for implanting the device provides potential advantages over existing methods. The inventive method may be safer and more effective than its alternatives. The access opening is smaller than alternative methods. The anterior access path is less obstructed by nerves, vessels and other delicate anatomy. Thus, the danger of injuring the surrounding anatomy is reduced. The smaller access opening reduces the chance of infection and bleeding.

The method of the invention stabilizes the lumbo-sacral junction from an anterior abdominal and/or retroperitoneal approach. The method involves anteriorly exposing the L5-S1 junction. The surgeon then removes sufficient disc material. The L5 anchor 10 is placed using methods taught by the Kuslich K-Centrum® patent. The lower bone anchor 12 and rod 22 are interfaced with S1 18 using screws 14a and 14b other fixation means, directed through the holes 24a and 24b in the lower bone anchor 12 to secure the lower bone anchor 12 to the sacral bone. The screws 14a and 14b are directed laterally and somewhat inferiorly into the ala 20 of the sacrum 18, just posterior to the anterior cortex of the alar, the strongest bone in the sacrum. The L5-S1 junction may then be compressed together by postural or other mechanical means, after which the rod is secured to the L5 anchor.

The present invention may be embodied in other specific forms without departing from the spirit or essential attributes thereof; and it is, therefore, desired that the present embodiment be considered in all respects as illustrative and not restrictive, reference being made to the appended claims rather than to the foregoing description to indicate the scope of the invention.

1. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor adapted for interfacing with the S1 sacral bone; and
an angled intermedullary rod operably connected to the upper and lower bone anchors wherein an angle of the intermedullary rod relative to a longitudinal axis of the spinal column replicates a desired angle between the L5 vertebra and the S1 sacral bone.

2. The apparatus of claim 1 wherein the lower bone anchor is a walled enclosure.

3. The apparatus of claim 2 wherein at least one of the walls of the lower bone anchor is angled to replicate a desired angle between the L5 vertebra and the S1 sacral bone.

4. The apparatus of claim 1 wherein the lower bone anchor is a plate.

5. The apparatus of claim 1 wherein the lower bone anchor is Y-shaped.

6. The apparatus of claim 1 wherein the lower bone anchor is adapted to contain fill material.

7. The apparatus of claim 1 wherein at least one surface of the lower bone anchor is treated to improve an interface with the S1 sacral bone.

8. The apparatus of claim 1 wherein at least one surface of the lower bone anchor is treated to improve bone-ingrowth potential.

9. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor adapted for interfacing with the S1 sacral bone and having a proximal end and a distal end, the distal end secured into the S1 sacral bone such that the proximal end not protrude above an anterior surface of the S1 sacral bone; and
an intermedullary rod operably connected to the upper and lower bone anchors.

10. The apparatus of claim 9 wherein the lower bone anchor is a walled enclosure.

11. The apparatus of claim 10 wherein at least one of the walls of the lower bone anchor is angled to replicate a desired angle between the L5 vertebra and the S1 sacral bone.

12. The apparatus of claim 9 wherein the lower bone anchor is a plate.

13. The apparatus of claim 9 wherein the lower bone anchor is Y-shaped.

14. The apparatus of claim 9 wherein the lower bone anchor is adapted to contain fill material.

15. The apparatus of claim 9 wherein at least one surface of the lower bone anchor is treated to improve the interface with the S1 sacral bone.

16. The apparatus of claim 9 wherein at least one surface of the lower bone anchor is treated to improve bone-ingrowth potential.

17. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor adapted for interfacing with the S1 sacral bone and having at least one variable angle socket, each socket adapted to receive a fastener to secure into the S1 sacral bone; and
an intermedullary rod operably connected to the upper and lower bone anchors.

18. The apparatus of claim 17 wherein the lower bone anchor is a walled enclosure.

19. The apparatus of claim 18 wherein at least one of the walls of the lower bone anchor is angled to replicate a desired angle between the L5 vertebra and the S1 sacral bone.

20. The apparatus of claim 17 wherein the lower bone anchor is a plate.

21. The apparatus of claim 17 wherein the lower bone anchor is Y-shaped.

22. The apparatus of claim 17 wherein the lower bone anchor is adapted to contain fill material.

23. The apparatus of claim 17 wherein at least one surface of the lower bone anchor is treated to improve the interface with the S1 sacral bone.

24. The apparatus of claim 17 wherein at least one surface of the lower bone anchor is treated to improve bone-ingrowth potential.

25. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor adapted for interfacing with the S1 sacral bone; and
an angled intermedullary rod means for operably connecting the upper and lower bone anchors wherein an angle of the intermedullary rod relative to a longitudinal axis of the spinal column replicates a desired angle between the L5 vertebra and the S1 sacral bone.

26. The apparatus of claim 25 wherein the lower bone anchor is a walled enclosure.

27. The apparatus of claim 26 wherein at least one of the walls of the lower bone anchor is angled to replicate a desired angle between the L5 vertebra and the S1 sacral bone.

28. The apparatus of claim 25 wherein the lower bone anchor is a plate.

29. The apparatus of claim 25 wherein the lower bone anchor is Y-shaped.

30. The apparatus of claim 25 wherein the lower bone anchor is adapted to contain fill material.

31. The apparatus of claim 25 wherein at least one surface of the lower bone anchor is treated to improve the interface with the S1 sacral bone.

32. The apparatus of claim 25 wherein at least one surface of the lower bone anchor is treated to improve bone-ingrowth potential.

33. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor means for interfacing with the S1 sacral bone and having a proximal end and a distal end, the distal end secured into the S1 sacral bone such that the proximal end does not protrude above an anterior surface of the S1 sacral bone; and
an intermedullary rod operably connected to the upper and lower bone anchors

34. The apparatus of claim 33 wherein the lower bone anchor is a walled enclosure.

35. The apparatus of claim 34 wherein at least one of the walls of the lower bone anchor are angled replicate a desired angle between the L5 vertebra and the S1 sacral bone.

36. The apparatus of claim 33 wherein the lower bone anchor is a plate.

37. The apparatus of claim 33 wherein the lower bone anchor is Y-shaped.

38. The apparatus of claim 33 wherein the lower bone anchor is adapted to contain fill material.

39. The apparatus of claim 33 wherein at least one surface of the lower bone anchor is treated to improve the interface with the S1 sacral bone.

40. The apparatus of claim 33 wherein at least one surface of the lower bone anchor is treated to improve bone-ingrowth potential.

41. A vertebral anchor apparatus for use between a lowermost vertebra (L5) and a sacral bone (S1) in the spinal column of a mammal, the apparatus comprising:
an upper bone anchor adapted for interfacing with the L5 vertebra;
a lower bone anchor adapted for interfacing with the S1 sacral bone and having at least one means for receiving a fastener at variable angles; and
an intermedullary rod operably connected to the upper and lower bone anchors.

42. The apparatus of claim 41 wherein the lower bone anchor is a walled enclosure.

43. The apparatus of claim 42 wherein at least one of the walls of the lower bone anchor are angled replicate a desired angle between the L5 vertebra and the S1 sacral bone.

44. The apparatus of claim 41 wherein the lower bone anchor is a plate.

45. The apparatus of claim 41 wherein the lower bone anchor is Y-shaped.

46. The apparatus of claim 41 wherein the lower bone anchor is adapted to contain fill material.

47. The apparatus of claim 41 wherein at least one surface of the lower bone anchor is treated to improve the interface with the S1 sacral bone.

48. The apparatus of claim 41 wherein at least one surface of the lower bone anchor is treated to improve bone-ingrowth potential.

49. A method for stabilizing the lowermost vertebra (L5) and a sacral bone (S1) in the spinal column of a mammal comprising:
anteriorly exposing the lumbo-sacral junction,
removing disc material,
interfacing an upper bone anchor with the L5 vertebra,
interfacing a lower bone anchor with the S1 sacral bone,
operably connecting an angled intermedullary rod securing the upper bone anchor to the lower bone anchor, such that an angle of the intermedullary rod relative to a longitudinal axis of the spinal column replicates a desired angle between the L5 vertebra and the S1 sacral bone.

50. The method of claim 49 further comprising the step of introducing fill material into a cavity in the lower bone anchor.

51. A method for stabilizing the lowermost vertebra (L5) and a sacral bone (S1) in the spinal column of a mammal comprising:
anteriorly exposing the lumbo-sacral junction;
removing disc material;
interfacing an upper bone anchor with the L5 vertebra;
interfacing a lower bone anchor having a proximal end and a distal end with the S1 sacral bone such that the proximal end does not protrude above an anterior surface of the S1 sacral bone; and
operably connecting an intermedullary rod securing the upper bone anchor to the lower bone anchor.

52. The method of claim 51 further comprising the step of introducing fill material into a cavity in the lower bone anchor.

53. A method for stabilizing the lowermost vertebra (L5) and a sacral bone (S1) in the spinal column of a mammal comprising:
anteriorly exposing the lumbo-sacral junction,
removing disc material,
interfacing an upper bone anchor with the L5 vertebra,
interfacing a lower bone anchor with the S1 sacral bone, the lower bone anchor having at least one variable angle socket adapted for insertion of a sacral attachment fastener,
operably positioning an intermedullary rod securing the upper bone anchor to the lower bone anchor,
inserting the sacral attachment fastener through the variable angle sockets into the ala of the sacrum, and
securing the lower bone anchor to the sacral bone with the sacral attachment fastener.

54. The method of claim 53 further comprising the step of introducing fill material into a cavity in the lower bone anchor.
